(19) United States
(12) Patent Application Publication
(10) Pub. No.: US 2025/0169976 A1
(43) Pub. Date: May 29, 2025

(54) EXTERNAL ANKLE BRACE

(71) Applicant: TayCo Brace, Inc., South Bend, IN (US)

(72) Inventors: Frederick John Ferlic, South Bend, IN (US); Mike Bean, South Bend, IN (US)

(21) Appl. No.: 18/819,076

(22) Filed: Aug. 29, 2024

Related U.S. Application Data
(63) Continuation of application No. 17/399,562, filed on Aug. 11, 2021, now abandoned.
Application No. 17/399,562 is a division of application No. 16/374,865, filed on Apr. 4, 2019, now abandoned.
Application No. 16/374,865 is a continuation-in-part of application No. 15/074,339, filed on Mar. 18, 2016, now abandoned.
(60) Provisional application No. 62/135,823, filed on Mar. 20, 2015.

(51) Int. Cl. A61F 5/01

(52) U.S. Cl. CPC A61F 5/0127; A61F 2005/0172

(57) ABSTRACT

An external ankle brace including a rigid heel enclosure having a rear portion and a forward portion, the rear portion configured to receive and at least partially encircle the heel portion of the shoe, the forward portion having a medial sidewall and a lateral sidewall for collectively and concurrently at least partially encircling the sides of the shoe concurrent with the rear portion connecting the medial and lateral sidewalls to collectively at least partially encircle the side. The ankle brace includes a lateral upright extension selectively perpendicular to the rigid heel enclosure and pivotally attached to the lateral sidewall, the lateral upright extension including a lateral reinforcing component and a medial upright extension selectively perpendicular to the rigid heel enclosure and pivotally attached to the medial sidewall, the medial upright extension including a medial reinforcing component.

### CROSS-REFERENCE TO RELATED APPLICATIONS

This application is a continuation application of U.S. patent application Ser. No. 17/399,562, filed Aug. 11, 2021, which is a divisional application of U.S. patent application Ser. No. 16/374,865, filed Apr. 4, 2019, which is a continuation-in-part of U.S. patent application Ser. No. 15/074,339, filed 18 Mar. 2016, which claims priority from U.S. Provisional Patent Application Ser. No. 62/135,823, filed 20 Mar. 2015, the entirety of each of which is hereby incorporated by reference for all purposes.

### TECHNICAL FIELD

The disclosure pertains generally to preventative and rehabilitative equipment, and more particularly to an ankle brace.

### BACKGROUND

In the world of sports, ankle injuries are among the most common cause of lost playing time in a sporting career, with a typical ankle injury leaving the athlete out of competition for up to a month. Ankle sprains occur when there is a rapid shifting of weight from one direction to another. The force generated from the movement causes the foot to roll either inwards, which is known as inversion rotation; or outwards, which is known as eversion rotation. Both the inversion and eversion motion of the ankle cause the ligaments on the outside of the ankle to stretch or tear depending on the force that was generated during the movement.

Current braces vary from woven fabric that acts as a glove and wraps around the ankle, to rigid plastic uprights that are strapped around the ankle. The woven fabric braces typically are made of a thin fabric that envelope the ankle and are laced together to support the ankle from both inversion and eversion rotation. The main drawback with these types of braces is that the material lacks the resistance to prevent the ankle from rolling under intense forces. Further, fabric braces also have to be worn within the shoe, which causes the shoe to fit tighter or, in some cases, forces the user to move up a shoe size in order to wear the brace. In terms of the rigid uprights braces, these braces are typically much heavier than the fabric braces and also much larger. Fitting a rigid brace into a tight shoe almost never works, which forces the user to move up to the next shoe size to accommodate for the bulkiness of the brace. When the user moves up a shoe size, the shoe is no longer sized correctly for the foot and thus loses a portion of its intended use and purpose. These braces leave the user at risk for further injury because either the brace isn't strong enough to support the ankle or the shoe isn't fitted properly to the foot.

### SUMMARY

In an aspect, an external ankle brace for selectively restricting movement of an ankle in at least one of a first direction and a rotation direction, and selectively permitting movement of the ankle in a second direction is provided. The external ankle brace is disposed on the exterior of a shoe. The shoe has a heel portion, a toe portion longitudinally spaced from the heel portion, a sole, and oppositely disposed sides. The external ankle brace comprises a rigid heel enclosure having a rear portion and a forward portion. The rear portion is configured to receive and at least partially encircle the heel portion of the shoe. The forward portion has a medial sidewall and a lateral sidewall for collectively and concurrently at least partially encircling the sides of the shoe concurrent with the rear portion connecting the medial and lateral sidewalls to collectively at least partially encircle the side, and fully encircle the heel portion, of the shoe. The lateral and medial sidewalls each extend from the rear portion toward a toe of a wearer's foot and each extend beyond a talus of the wearer's foot. A lateral upright extension is selectively perpendicular to the rigid heel enclosure and is pivotally attached to the lateral sidewall. The lateral upright extension includes a lateral reinforcing strut. A medial upright extension is selectively perpendicular to the rigid heel enclosure and is pivotally attached to the medial sidewall. The medial upright extension includes a medial reinforcing strut. A lower fastening system comprises at least one lower connecting strap for connecting the lateral sidewall to the medial sidewall and extending underneath the sole of the shoe. An upper fastening system comprises at least one upper connecting strap for selectively connecting the lateral sidewall to the medial sidewall across the top of the shoe. The upper connecting strap is located longitudinally between the lower connecting strap and the lateral and medial upright extensions.

### BRIEF DESCRIPTION OF THE DRAWINGS

For a better understanding, reference may be made to the accompanying drawings, in which:

FIG. 1 is a lateral side view showing a first aspect of the external ankle brace with an athletic shoe.

FIG. 2 is a perspective view of the external ankle brace of FIG. 1 from

the medial side.

FIG. 3 is a perspective view showing the underside of the external ankle brace of FIG. 1.

FIG. 4 is a perspective view of the external ankle brace of FIG. 1 from the lateral side.

FIG. 5 is a top view of the external ankle brace of FIG. 1.

FIG. 6 is a rear view of the external ankle brace of FIG. 1.

FIG. 7 is a lateral side view showing a second aspect of the external ankle brace, in a first configuration, with an athletic shoe.

FIG. 8 is a lateral side view showing a second aspect of the external ankle brace, in a second configuration, with an athletic shoe.

FIG. 9 is a perspective rear view of a component of the external athletic braces of FIGS. 7-8.

FIG. 10 is a partial perspective front view of the component of FIG. 9.

FIG. 11 is a partial front view of the component of FIG. 9.

FIG. 12 is a side view of the component of FIG. 9.

FIG. 13 is a bottom view of the component of FIG. 9.

### DETAILED DESCRIPTION

Unless defined otherwise, all technical and scientific terms used herein have the same meaning as is commonly understood by one of skill in the art to which the present disclosure pertains.

In the context of the present disclosure, the singular forms “a,” “an” and “the” can include the plural forms as well, unless the context clearly indicates otherwise. It will be further understood that the terms “comprises” and/or “comprising,” as used herein, can specify the presence of stated features, steps, operations, elements, and/or components, but do not preclude the presence or addition of one or more other features, steps, operations, elements, components, and/or groups thereof.

As used herein, the term “and/or” can include any and all combinations of one or more of the associated listed items.

As used herein, the term “and/or” can include any and all combinations of one or more of the associated listed items.

It will be understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

Spatially relative terms, such as “under,” “below,” “lower,” “over,” “upper” and the like, may be used herein for ease of description to describe one element or feature's relationship to another element(s) or feature(s) as illustrated in the figures. It will be understood that the spatially relative terms can encompass different orientations of the apparatus in use or operation in addition to the orientation depicted in the figures. For example, if the apparatus in the figures is inverted, elements described as “under” or “beneath” other elements or features would then be oriented “over” the other elements or features.

It will be understood that, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure.

A “wearer” or “user”, as described herein, is a person who has the external ankle brace on his or her own foot/ankle.

Ankle injuries are among the most common cause of lost playing time in a sporting career and although there are current preventative solutions, those current braces leave the user at risk for further injury because either the brace isn't strong enough to support the ankle or the shoe isn't fitted properly to the foot since “inside the shoe” braces tend to force the user to use a bigger shoe size. In addition, outside the athletic context, ankle braces can be used by the general population while recovering from ankle injury or attempting to prevent reinjury, to wear while going about daily activities.

The present disclosure provides a rigid support and a much faster application time, all without compromising the fit of the shoe. Essentially, the external ankle brace described herein assists anyone recovering from an ankle injury and/or seeking to avoid injury to limit inversion/eversion and rotation, and can also selectively limit plantar flexion/dorsiflexion. This at least partial motion limiting can help an ankle to heal or avoid (re) injury; thus, the patient can return to function and normal daily activities (including athletic activities, as desired) without the need for specialized or temporary-use footwear (e.g., a larger shoe on the braced side). The external ankle brace of the present disclosure may provide superior stability to an internal ankle brace and improved functionality over known solutions, such as a walking boot or internal ankle foot orthoses.

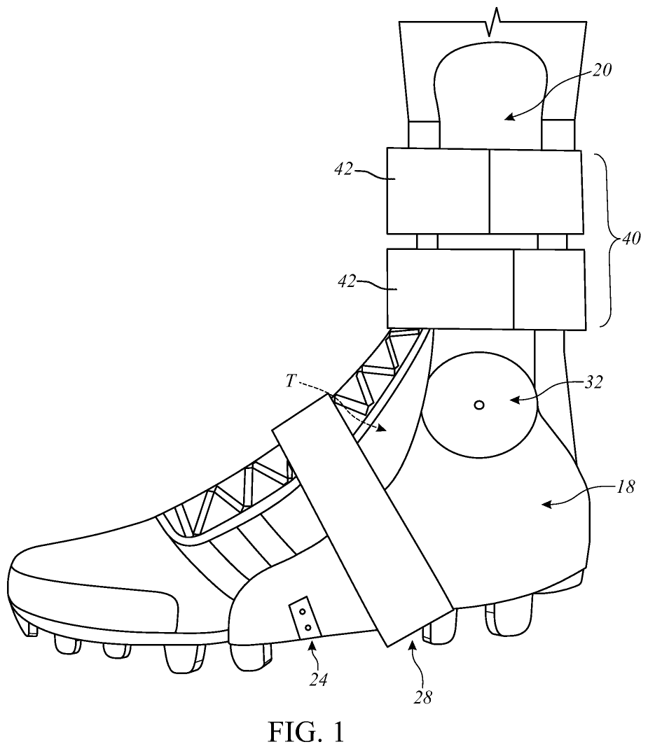

The present disclosure relates to an external ankle brace that is adapted to fit around a shoe to prevent and minimize injury to an ankle. The shoe has a heel portion, a toe portion longitudinally spaced from the heel portion, a sole, and oppositely disposed sides. The term “longitudinal” is used herein to reference a direction oriented along the foot between the heel and toe. The interaction between the external ankle brace and the shoe can be seen in FIG. 1.

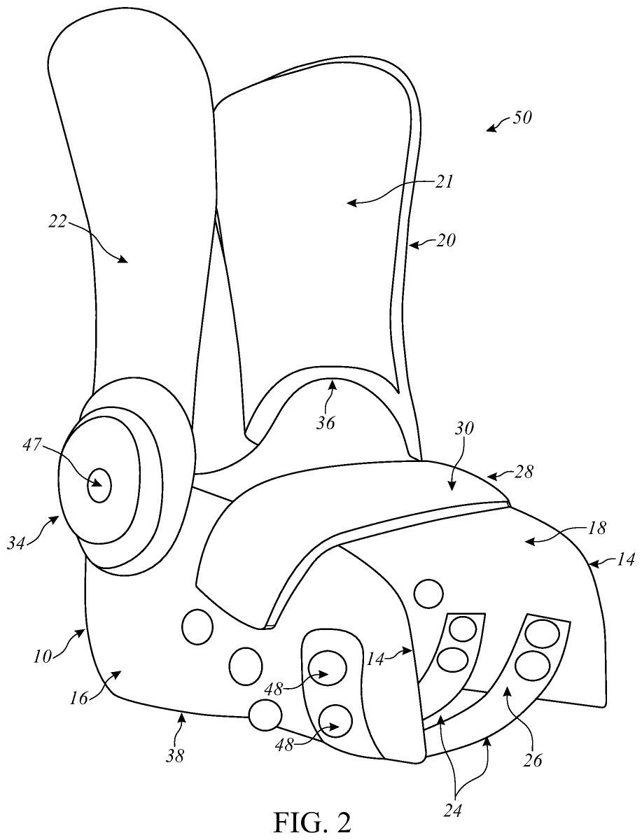

The external ankle brace of the present disclosure is generally indicated at 50 in FIG. 2. The external ankle brace 50 includes a rigid heel enclosure 10, a lateral upright extension 20, a medial upright extension 22, a lower fastening system 24, and an upper fastening system 28.

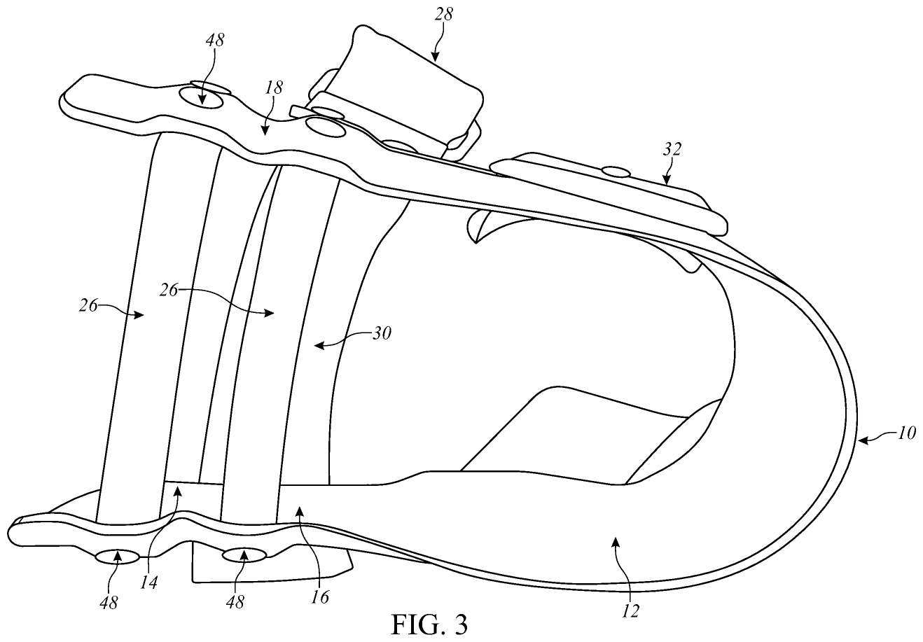

The rigid heel enclosure 10 has a rear portion 12 (FIG. 3), for receiving and at least partially encircling the heel of the shoe, and a forward portion 14, for surrounding, e.g., collectively and concurrently at least partially surrounding, the sides of the shoe. The forward portion 14 at least partially encircles the sides of the shoe concurrent with the rear portion 12 connecting the medial and lateral sidewalls 18 and 16 to collectively at least partially encircle the side, and fully encircle the heel, of the shoe. The heel enclosure 10 may be at least partially made from rigid plastic pieces and/or any other suitable material. The forward portion 14 further includes a medial sidewall 16 and a lateral sidewall 18, which may also be at least partially made from rigid plastic pieces and/or any other suitable material.

As shown in the Figures, it is contemplated that the lateral and/or medial sidewalls 16 and 18 may each extend from the rear portion 12 in a longitudinal direction toward a toe of the wearer's foot and may each extend beyond a talus (shown as “T” in FIG. 1) of the wearer's foot—i.e., may be located lateral/medial to the lace portion of the shoe. (It is recognized that a particular wearer may have nonstandard bone structure to which this “landmark” would not apply—this description presumes a foot structure which is anatomically normal, in which the talus marks the “inflection point” at which the lower leg turns into the top of the foot.) Stated differently, the rigid heel enclosure 10 has a rear portion 12, and a forward portion 14 partially located on both sides of the heel, one side of the forward portion 14 comprising the medial sidewall 16 and an other side of the forward portion 14 comprising the lateral sidewall 18. Each of the medial and lateral sidewalls 16 and 18 extends at least a predetermined amount of the longitudinal distance from a pivot point on the rigid heel enclosure 12 forward from heel toward toe. The medial and lateral sidewalls 16 and 18 run beside (and thus at least partially surround) a predetermined percentage of a total longitudinal length of the user's shoe that is in the range of 5-40%, more particularly about 10-35%, and more particularly about 20-30% of the longitudinal distance between the user's ankle and the end of the user's toe. It is contemplated that an external ankle brace 50 will, for most use environments, include medial and lateral sidewalls 16 and 18 that extend beyond the talus T (as opposed to a straight “stirrup” on the sides of the ankle with little/no extension), to accommodate placement of a lower connecting strap 26 that fits under the shoe's arch gap.

The rigid heel enclosure 10 also has an upper end 36 (FIG. 2) for receiving the upright extensions 20 and 22, and a lower end 38 for surrounding the bottom of the shoe. The rigid heel enclosure 10 can be configured as desired, such as by orienting the lower end 38 in a substantially “straight” non-contoured configuration (parallel to, and fitting closely about, the base of the shoe), and/or the upper end 36 including a curved contour as shown in FIG. 2 for mechanical, manufacturing efficiency, aesthetic, and/or any other considerations.

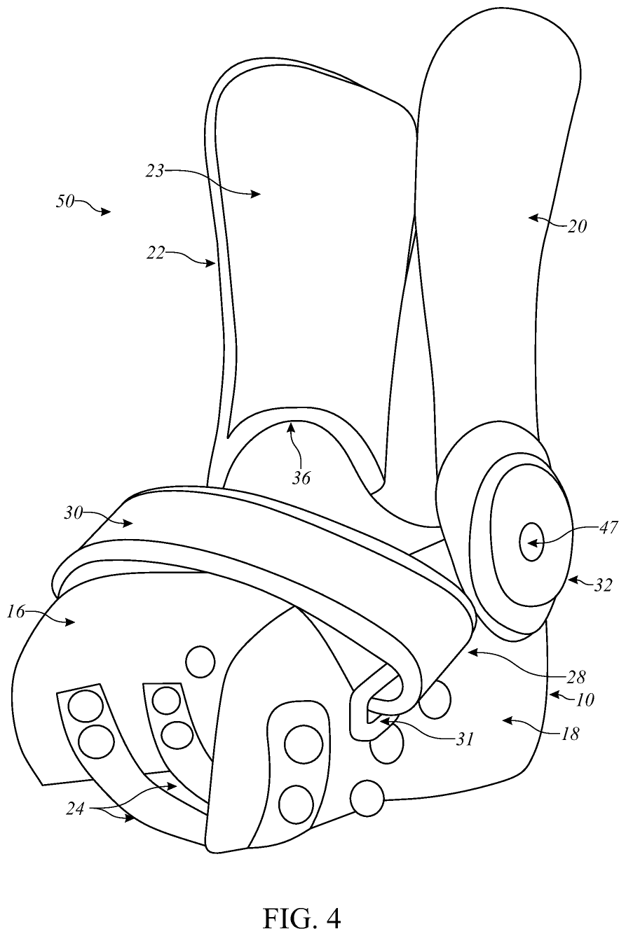

The lateral upright extension 20 is oriented generally selectively perpendicular to at least the lateral sidewall 18 of the rigid heel enclosure 10 and is pivotally attached to the lateral sidewall 18 at the upper end 36 by a lateral ankle joint 32 (FIG. 4). The joint allows the lateral upright extension 20 to rotate during motion giving the external ankle brace a less restrictive feel compared to previous braces—thus, the lateral upright extension 20 is perpendicular to the lateral sidewall 18 during certain portions of the pivoting process, such as when the ankle is in a neutral position, in neither planar flexion nor dorsiflexion.

In other words, the lateral sidewall 18 is both substantially planar in a vertical fashion (up and down) and substantially planar in a longitudinal fashion (back of shoe to front of shoe), including some minor contours to fit the curves of a shoe and/or a user. The lateral upright extension 20 selectively pivots forward and backward to allow for plantar flexion and dorsiflexion while still providing inversion/eversion and rotational support. As desired, the lateral upright extension 20 may be configured for permanent or temporary fixation with respect to the lateral sidewall 18, which maintains perpendicularity therebetween and does not allow for plantar flexion or dorsiflexion. When the fixation screw (or screws) are removed as discussed below to reverse a temporary fixation situation, then the lateral and medial upright extensions 20 and 22 can move forward and backward to allow for plantar flexion and dorsiflexion.

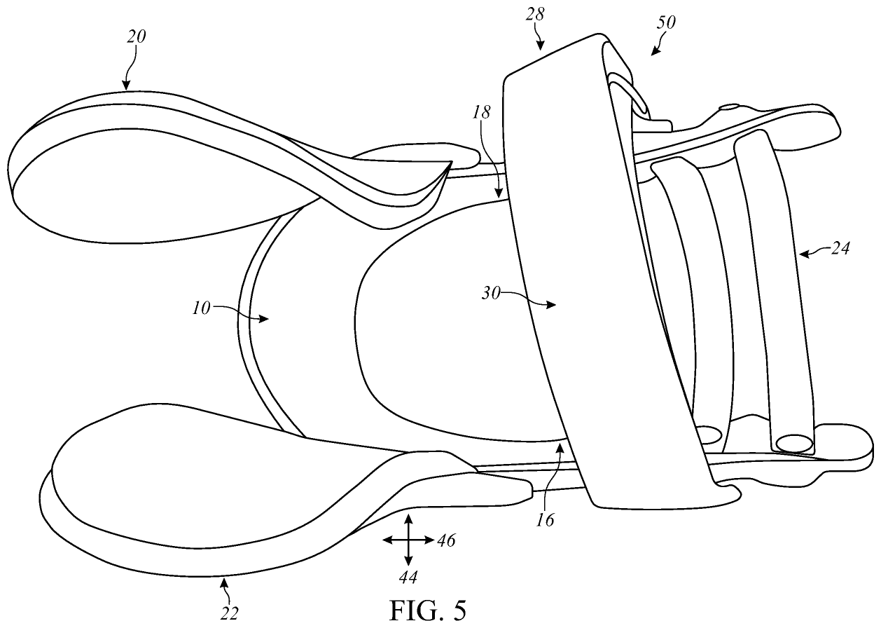

The lateral upright extension 20 may be made from plastic and/or any other suitable material. The lateral ankle joint 32 includes a fastener 47 and allows the lateral upright extension 20 to rotate relative to the lateral sidewall 18. Although the current embodiment uses at least one screw as the fastener 47, one having ordinary skill in the art will appreciate that a pivot hinge, hex nut, revolving joint, Chicago screw, or any other suitable member could be used to allow the joint to pivot. As shown in FIG. 5, the lateral upright extension 20 may have a concave shape for increased comfort for the user. The lateral upright extension 20 can also include foam or other suitable padding on the interior side 21 (FIG. 2) of the lateral upright extension 20 to increase comfort and to allow a better fit for the user.

The medial upright extension 22 is oriented generally selectively perpendicular to at least the medial sidewall 16 of the rigid heel enclosure 10 and is pivotally attached to the medial sidewall 16 at the upper end 36 by a medial ankle joint 34, with the perpendicularity and pivotal properties being similar to those of the lateral upright extension 20. The medial upright extension 22 may be made of rigid plastic and/or any other suitable material. The medial ankle joint 34 has a fastener 47 and allows the medial upright extension 22 to rotate relative to the medial sidewall 16—thus, the medial upright extension 22 is perpendicular to the medial sidewall 16 during certain portions of the pivoting process.

To adjust for anatomical positioning of the ankle, the medial ankle joint 34 may be positioned somewhat closer to the upper end 36 than the position of the lateral ankle joint 32, since the medial malleolus (that bone that protrudes from the inside of the user's ankle) may be slightly higher (more cephalad) than the lateral malleolus (the bone that protrudes from the outside of the ankle), in most anatomically normal wearers. The pivot points between the lateral and medial upright extensions 20 and 22, and their respective lateral and medial sidewalls 18 and 16 on each side of the external ankle brace 50 could be desirably aligned with the medial-higher anatomy of the body, although it is contemplated that versions of the external ankle brace 50 could have equal-medial-lateral or medial-lower pivot points for any reason(s), such as, but not limited to, manufacturing considerations or the anatomy of a particular user.

In addition, the medial ankle joint 34 of the external ankle brace 50 may be offset, as desired from a 180-degree directly opposing position relative to the lateral ankle joint 32. For example, the medial ankle joint 34 may be rotated in the range of about 5-20 degrees, and more particularly about 10-15 degrees, forward (towards the toes) from a 180 degree position relative to the lateral ankle joint 32. Similar to the ankle joint height discussed immediately previously, this is anatomically correct. To clarify, imagine a circle that is parallel to the ground (on the same plane). Instead of having the medial and lateral ankle joints 34 and 32 being 180 degrees apart on that circle, the medial ankle joint 34 could be moved (and rotated to keep facing the center of the circle, if desired), some distance toward the toes along that circle because the medial malleolus is closer to the toes than is the lateral malleolus. It is contemplated that versions of the external ankle brace 50 could instead have 180-degree-spaced or lateral-forward pivot points for any reason(s), such as, but not limited to, manufacturing considerations or the anatomy of a particular user.

Although the current embodiment uses at least one screw as the fastener 47, one having ordinary skill in the art will appreciate that a pivot hinge, hex nut, revolving joint, Chicago screw, or any other suitable member could be used to allow the joint to pivot. As shown in FIG. 5, the medial upright extension 22 may have a concave shape for increased comfort for the user. The medial upright extension 22 can also include foam or other padding on the interior side 23 (FIG. 4) of the medial upright extension to increase comfort and to allow a better fit for the user.

The lower fastening system 24 has at least one lower connecting strap 26 and at least one strap fastener 48 for connecting the lateral sidewall 18 to the medial sidewall 16 (FIG. 2) and extending underneath the sole of the shoe. Although the current embodiment uses a rubber strap, one having ordinary skill in the art would appreciate that plastic, nylon, or any other suitable strap type that is commonly known in the art could be used. Similarly, although the current embodiment uses rivets to fasten the straps to each of the lateral and medial sidewalls 18 and 16 respectively, any other fastener could be used.

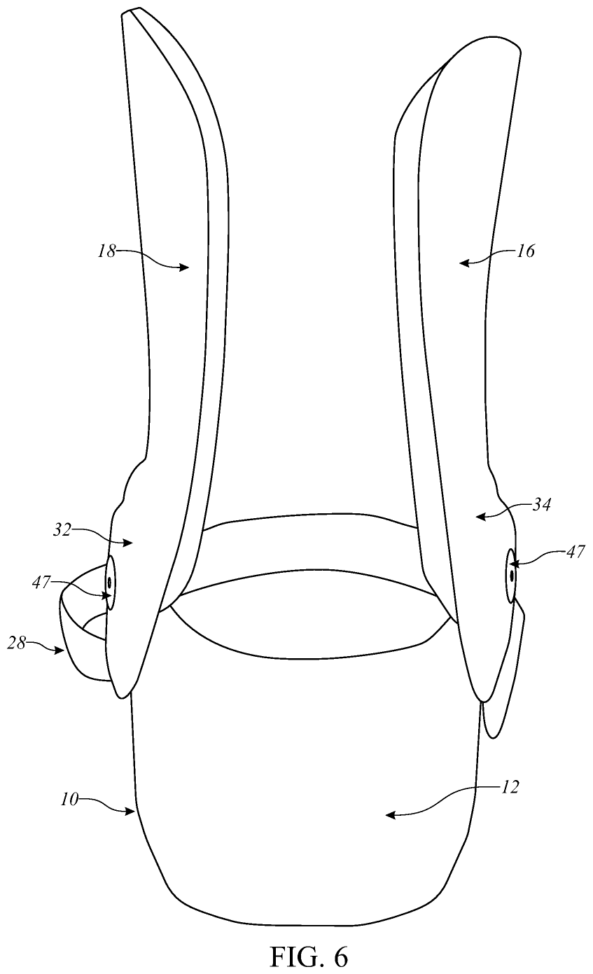

The upper fastening system 28 has at least one upper connecting strap 30 for selectively connecting the lateral sidewall 18 to the medial sidewall 16 while passing over the top of the shoe. The upper fastening system 28 further includes a D-ring 31 which is fixed on the lateral sidewall. A hook and loop fastener type upper connecting strap 30 is fixed to the medial sidewall and is looped through the D-ring 31 to overlaps back itself. This allows for an adjustable fastening system to accommodate various sizes without compromising support. The term “hook and loop fastener” is used herein to reference a type of fastening device such as, but not limited to, VELCRO® brand fasteners, available from Velcro USA Inc. & Velcro Group Corporation of Manchester, New Hampshire. Although the current embodiment uses a hook and loop fastener upper connecting strap 30 to removeably connect the sidewalls 16 and 18 and/or adjust the tightness of their mutual connection across the wearer's instep, one having ordinary skill in the art would appreciate that any kind of removable and/or adjustable strap can be used. Similarly, although the embodiment of FIGS. 1-6 only uses one upper connecting strap 30, any number of straps can be used to removeably and/or adjustably connect the sidewalls 16 and 18 over the top of the shoe, as will be discussed further below. As shown in FIGS. 1-5, the upper connecting strap 30 is located longitudinally between the lower connecting strap 26 and the lateral and medial upright extensions 20 and 22, though one of ordinary skill in the art could readily configure an external ankle brace 50 having lower connecting strap(s) 26 in a desired location to fit a particular wearer, a selected shoe model, a certain cleat spacing, or for any other reason. The term “longitudinally between” is used herein to relate to the relative positions of two or more structures in the longitudinal direction—the two or more structures need not intersect, in whole or part, the same longitudinal line as one another but could be offset along different, substantially parallel longitudinal lines.

As shown in FIG. 5, the external ankle brace 50 restricts movement of the ankle in the first directions indicated by arrows 44 (the eversion and inversion directions) and permits ankle movement in the second directions indicated by arrows 46 (the plantar flexion and dorsiflexion directions). The external ankle brace 50 also restricts rotation of the ankle (“windshield wiper” motion of the toes with a stationary heel, or vice versa.

The external ankle brace 50 may include an upright fastening system 40 (FIG. 1), which would have at least one upright connecting strap 42 for selectively connecting the lateral upright extension 20 to the medial upright extension 22 above the ankle. This upright connecting strap 42 could include hook and loop fastener or any other type of strap that would allow for an adjustable and/or removable connection.

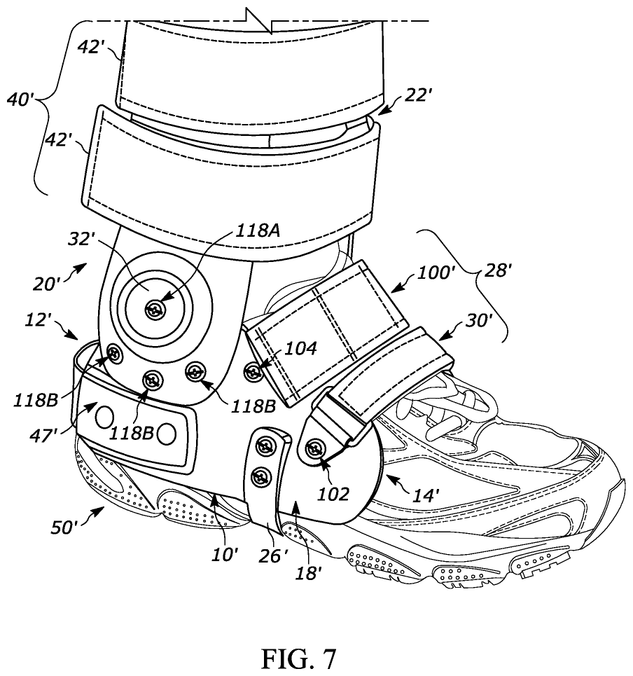

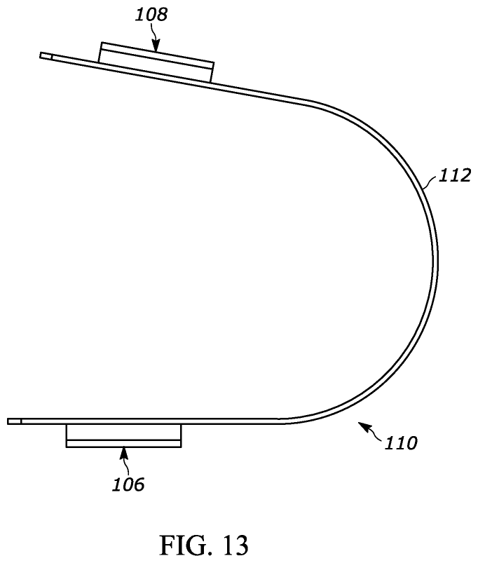

FIGS. 7-13 illustrate a second embodiment of an external ankle brace 50′. The external ankle brace 50′ of FIGS. 7-13 is similar to the external ankle brace 50 of FIGS. 1-6 and therefore, structures of FIGS. 7-13 that are the same as or similar to those described with reference to FIGS. 1-6 will be shown and/or described as having the same reference numbers with the addition of a “prime” mark. Description of common elements and operation similar to those in the previously described first embodiment will not be repeated with respect to the second embodiment or vice versa, but should instead be considered to be incorporated below/above by reference as appropriate.

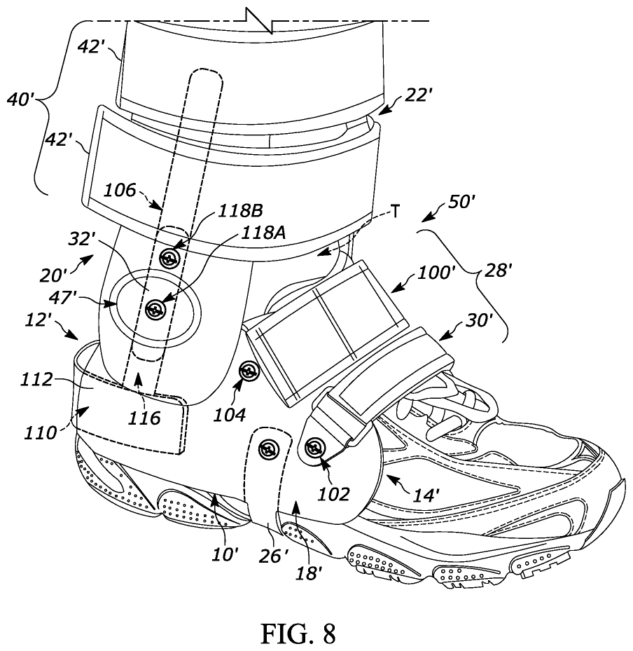

As shown in FIGS. 7-8, the external ankle brace 50′ may include an upright fastening system 40′ comprising at least one upright connecting strap 42′ (two shown) for selectively connecting the lateral upright extension 20′ to the medial upright extension 22′ above the ankle. It is contemplated that at least one of the upright connecting straps 42′ (or any other straps of the external ankle brace 50′) could have elastic properties to allow slight motion of the lateral and medial upright extensions 20′ and 22′ relative to one another as desired, as the external ankle brace 50′ is doffed and donned by the wearer.

Also as shown in FIGS. 7-8, the upper connecting strap 30′ is a first upper connecting strap 30′, and the upper fastening system 28′ includes a second upper connecting strap 100 for selectively connecting the lateral sidewall 18′ to the medial sidewall 16′ across the top of the shoe. The second upper connecting strap 100, when present, may be located longitudinally between the first upper connecting strap 30′ and the lateral and medial upright extensions 20′ and 22′. When rotation of the medial and lateral upright extensions 20 and 22 is “fixed” in a perpendicular position (an option discussed elsewhere herein), the second upper connecting strap 100 may be helpful to prevent plantar flexion and dorsiflexion, thus making the external ankle brace 50′ in this “non-pivoting mode” similar in overall stability to a fixed walking boot.

It is also contemplated that the first upper connecting strap 30′ (as shown in the Figures) could be omitted from the external ankle brace 50′, allowing the component shown and described herein as the second upper connecting strap 100 to serve as the (only) upper connecting strap, substantially in the position shown and described herein.

The second upper connecting strap 100 may be longitudinally wider than the first upper connecting strap 30′, as shown, for any reason. For example, in some use environments, the second upper connecting strap 100 will doing the majority of the support work (of the two upper connecting straps 100 and 30′) and be subject to the majority of the forces generated in-use. A wider strap in such circumstances distributes those forces and facilitates increased comfort for the wearer as opposed to a narrower strap (which might be more inclined to “cut in” at the edges to the wearer's foot/leg). Because the force is spread out by the wider strap, the wearer can use the external ankle brace 50′ for a long period of time without pain. The narrower first upper connecting strap 30′ is narrower, as shown, since there will not be enough room for another strap having a similar width to the second upper connecting strap 100 in many use configurations of the external ankle brace 50′.

As can be seen in at least FIG. 7, the first upper connecting strap 30′ may be permanently or adjustably attached to the respective lateral and/or medial sidewall 18′ or 16′ via a first upper fastener 102. As shown in the Figures, the first upper fastener 102 may attach the first upper connecting strap 30′ to an outer surface of the respective lateral and/or medial sidewall 18′ or 16′, such that the lateral and/or medial sidewall 18′ or 16′ is at least partially interposed between the body of the first upper connecting strap 30′ and the shoe.

Additionally, the second upper connecting strap 100, as shown in the Figures, may be permanently or adjustably attached to the respective lateral and/or medial sidewall 18′ or 16′ via a second upper fastener 104. As shown in the Figures, and in contrast to the first upper connecting strap 30′, the second upper fastener 104 attaches the second upper connecting strap 100 to an inner surface of the respective lateral and/or medial sidewall 18′ or 16′, such that the body of the second upper connecting strap 100 is at least partially interposed between the lateral and/or medial sidewall 18′ or 16′ and the shoe. In the case of the external ankle brace 50′ shown in FIGS. 7-13, this respective inner/outer fastening of the straps, at their respective positions, is considered to give a tighter and more secure fit of the external ankle brace 50′ around the wearer's shoe.

It is contemplated that one of ordinary skill in the art could similarly configure the strap placements and fastening types (number and kind of fasteners, inside/outside placement, and the like) for a particular use environment and to facilitate economical manufacture balanced with desired bracing results. For example, and as shown in FIGS. 7-8, the lower connecting strap 26′ of the external ankle brace 50′ may be located longitudinally between the first upper connecting strap 30′ and the lateral and medial upright extensions 20′ and 22′, and/or the second upper connecting strap 100 may be located longitudinally between the lower connecting strap 26′ and the lateral and medial upright extensions 20′ and 22′. As another example, in the case of the inside fastening of the second upper connecting strap 100, testing has shown that the malleability of the plastic of the external ankle brace 50′, and “lifting” of an externally-fastened upper connecting strap 100 away from the shoe by the respective lateral and/or medial sidewall 18′ or 16′, permit an undesirable degree of laxness in the fastening scheme for some use environments. That is, attaching the second upper connecting strap 100 to the inside of the lateral and medial sidewalls 18′ and 16′ may, in some use environments, assist with providing desired properties of fit, comfort, and stability. If the second upper connecting strap 100 were to be fastened outside the lateral and medial sidewalls 18′ and 16′, at the talus-adjacent position on the shoe shown in FIGS. 7-8, it may be more difficult to achieve a desired amount of comfort and stability for the wearer. As another example, in some use environments, the upper connecting strap 30′, 100 may include a variable length fastening operable for manual adjustment by a wearer to a predetermined length. As another example, the lower connecting strap 26′ may either include a variable length fastening operable for manual adjustment by a wearer to a predetermined length, or may have a constant length and not be manually adjustable by a wearer (e.g., to provide durability against a wearing surface of the shoe sole).

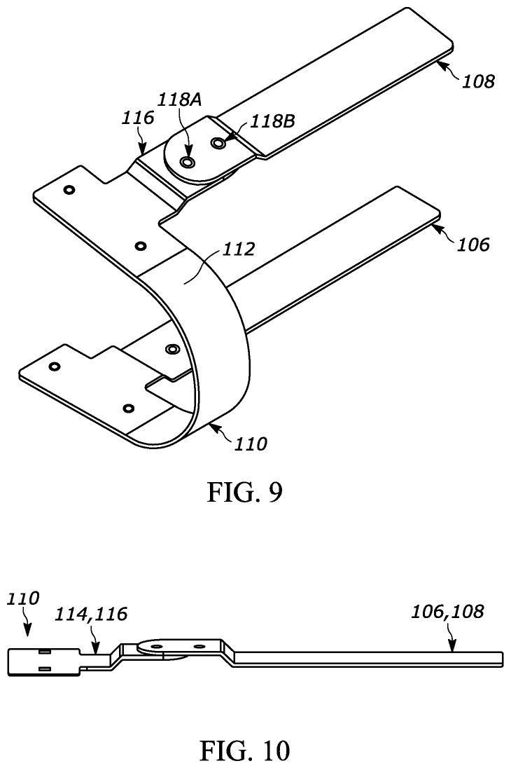

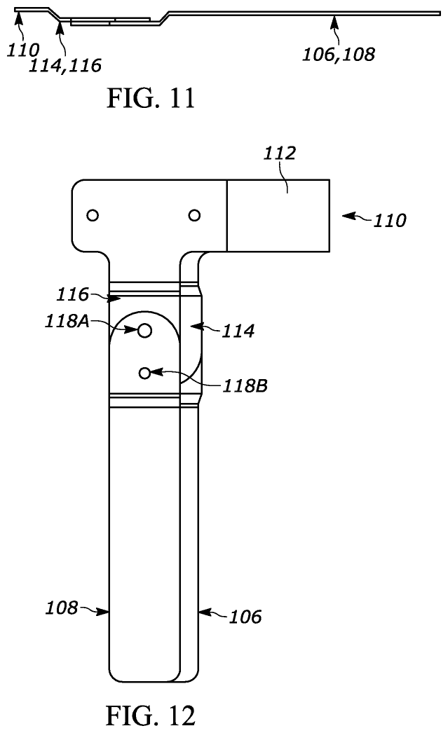

With reference now to FIGS. 8-13, the lateral upright extension 20′ may include a lateral reinforcing strut (shown in phantom view as 106 in FIG. 8), and the medial upright extension 22′ may include a medial reinforcing strut (shown as 108 in FIG. 9). The lateral and medial reinforcing struts 106 and 108, when present, may be malleable and configured to accept and maintain a nonplanar shape profile. A “nonplanar shape profile” is used herein to indicate that the component may be bent—manually and/or automatically—out of the substantially planar orientation shown for the lateral and medial reinforcing struts 106 and 108 in at least FIGS. 9 and 11. When the lateral and/or medial reinforcing struts 106 and 108 are in the nonplanar shape profile, they are configured to at least partially impart the nonplanar shape profile to the corresponding lateral or medial upright extension 20′ and 22′. In practical application, the lateral and/or medial reinforcing struts 106 and 108, and other, similar structures, can be used to help plastically and/or elastically deform the external ankle brace 50′, by a user/prescriber and/or machine, to fit the contours of a wearer's body in a desired manner. For example, if the wearer has very large or very small calf muscles, an orthotic or prosthetic assistance device, or any other feature that is different than that contemplated by a stock external ankle brace 50′, the external ankle brace 50′ can be configured (before, during, or after purchase/use) for that wearer or a like class of wearers.

The lateral and medial reinforcing struts 106 and 108 help with stability of the external ankle brace 50′ during use and may also facilitate custom fitting, or shaping the brace to the contours of an individual ankle and lower calf. Without the lateral and medial reinforcing struts 106 and 108, a prescriber would likely have to heat the plastic to mold it properly, and then it may not retain its shape under wear forces during use. The lateral and medial reinforcing struts 106 and 108 assist the respective lateral and medial upright extensions 20 and 22 in being shaped easily but then also holding a shape and providing stability.

It is contemplated that the lateral and medial upright extensions 20′ and 22′ may be made of a first material, and the lateral and medial reinforcing struts 106 and 108 may be made of a second material which is more ductile than the first material. For example, the first material may be a polymer and the second material may be a metal, such as, but not limited to, aluminum. It is also contemplated that the lateral and medial reinforcing struts 106 and 108 could be made from carbon-fiber and custom-manufactured to fit a particular user or class of users.

The lateral and medial upright extensions 20′ and 22′ may wholly encapsulate the lateral and medial reinforcing struts 106 and 108. This could be accomplished, for example, by the lateral and medial upright extensions 20′ and 22′ being molded around, or otherwise fabricated to encompass, the lateral and medial reinforcing struts 106 and 108. The material of the lateral and medial upright extensions 20′ and 22′ could be significantly larger in cross-sectional size than the respective lateral and medial reinforcing struts 106 and 108, or could instead be a relatively thin “skin” (e.g., a vinyl coating) interposed between the lateral and medial reinforcing struts 106 and 108 and the ambient space. Additionally, it is contemplated that the lateral and/or medial reinforcing struts 106 and 108 could be left bare to themselves serve directly as lateral and/or medial upright extensions 20′ and 22′.

With reference now to FIGS. 8-13, the rear portion 12′ may include a rear reinforcing strut 110. The rear reinforcing strut 110 may be malleable and configured to accept and maintain a nonplanar shape profile, to at least partially impart the nonplanar shape profile to the rear portion 12′, although it is contemplated that the rear reinforcing strut 110 could also or instead be relatively rigid and configured to maintain the lateral and medial reinforcing struts 106 and 108 in a predetermined spacing and mutual orientation relationship (e.g., to avoid “torqueing” or “twisting” of the lateral and medial upright extensions 20′ and 22′. despite any reconfiguring of the lateral and/or medial reinforcing struts 106 and 108).

As with the external ankle brace 50 of FIGS. 1-6, the rear portion 12′ of the external ankle brace 50′ of FIGS. 7-13 encircles the heel, but leaves the heel of the shoe exposed so that the user's shoe can directly contact the ground. The rear portion 12′ of the rigid heel enclosure 10′ is cut out to leave the bottom of the heel, and a portion of the side of the heel, of the shoe exposed. As depicted in at least FIG. 7, the back of the external ankle brace 50′ may fit or rest on the base of the heel counter of the shoe, which is not the base of the shoe. The external ankle brace 50′ does not touch the ground at the heel of the shoe and there is a gap (e.g., about a half inch, for some users) between the sole of the shoe and the rigid heel enclosure 10′.

Whether or not deformation of the rear reinforcing strut 110 is contemplated, the rear portion 12′ may be made of a first material and the rear reinforcing strut 110 may be made of a second material which is more ductile than the first material. For example, the first material may be a polymer and the second material may be a metal. For example, the first material may be a polymer and the second material may be a metal, such as, but not limited to, aluminum. It is also contemplated that the rear reinforcing strut 110 could be made from carbon-fiber and custom-manufactured to fit a particular user or class of users.

The rear portion 12′ may wholly encapsulate the rear reinforcing strut 110. The rear portion 12′ may be molded around the rear reinforcing strut 110. The material of the rear portion 12′ could be significantly larger in cross-sectional size than the rear reinforcing strut 110, or could instead be a relatively thin “skin” (e.g., a vinyl coating) interposed between the rear reinforcing strut 110 and the ambient space. Additionally, it is contemplated that the rear reinforcing strut 110 could be left bare to itself directly serve as a rear portion 12′. A material selection (e.g., a high-friction material) and/or surface treatment (e.g., knurling) may be used on the rear portion 12′ (or directly on the rear reinforcing strut 110, when serving as the rear portion 12′) to increase frictional surface area contacting the heel counter of the shoe to provide desired fixation to help restrict plantar flexion and dorsiflexion. (As with several components of the external ankle brace 50′, including, but not limited to, the lateral and medial upright extensions 20′ and 22′ and the rear and forward portions 12′ and 14′, it may be desirable to balance manufacturing considerations, friction of surfaces, area of surfaces, and weight of components in seeking comfort, stability, security/tightness, motion restriction, and usability for the wearer.)

The rear reinforcing strut 110 may include a curved rear strut body 112 extending around the heel portion of the shoe and lateral and medial strut stubs 114 and 116, respectively, extending substantially perpendicularly from the rear strut body 112, at opposed locations on the rear strut body 112. The lateral and medial reinforcing struts 106 and 108 are directly pivotally connected to the lateral and medial strut stubs 114 and 116, respectively. The lateral, medial, and/or rear reinforcing struts 106, 108, and 110, when present, may provide desired rigidity and/or strength, such as to permit a lower-profile size, to the respective lateral and medial upright extensions 20′ and 22′ and/or rear portion 12′.

As shown in the Figures, the rear reinforcing strut 110 may be directly pivotally connected to the lateral and medial reinforcing struts 106 and 108 in any desired manner. For example, at least one restraining bolt 118 may be connected to a chosen one of the lateral and medial upright extensions 20′ and 22′ and be operative to selectively restrict pivoting of the chosen upright extension 20′ and 22′ respective to a corresponding lateral or medial sidewall 18′ or 16′. In FIG. 7, this configuration is shown as a primary restraining bolt 118A and a plurality (three shown) of secondary restraining bolts 118B. The primary restraining bolt 118A in FIG. 7 is located at a pivot point of the lateral ankle joint 32′. The secondary restraining bolts 118B, when present, resist pivoting of the respective lateral or medial upright extension 20′ and 22′ with respect to a corresponding lateral or medial sidewall 18′ or 16′. Thus, restraining bolts 118A and/or 118B may be connected to a chosen one of the lateral and medial reinforcing struts 106 and 108 and may be operative to either assist/facilitate or selectively restrict pivoting of the chosen upright extension 20′ and 22′ respective to the rear reinforcing strut 112.

As another example, FIGS. 8-13 depict a configuration of the external ankle brace 50′ shown as having one restraining bolt 118A at a center pivot location of the lateral or medial ankle joint 32 and 34, and at least one second restraining bolt 118B at a location off-center from the pivoting point but still configured to selectively attach the respective lateral or medial upright extension 20′ or 22′ directly to a corresponding structure associated with the ankle joint 32 or 34 and thus substantially prevent relative pivoting therebetween. For example, and as shown in at least FIGS. 9-12, the second restraining bolt 118B may selectively mutually connect the lateral or medial strut stub 114 or 116 to a respective lateral or medial reinforcing strut 106 or 108.

It is contemplated, however, that one or more second restraining bolts 118B could instead be used at a center pivot location of the lateral or medial ankle joint 32 and 34, and at least one restraining bolt 118A could be placed at a location off-center from the pivoting point but still configured to selectively attach the respective lateral or medial upright extension 20′ or 22′ directly to a corresponding structure associated with the ankle joint 32 or 34 and thus substantially prevent relative pivoting therebetween as desired. In this alternate situation, the second restraining bolt 118B shown in FIG. 8 could be located for pivotal alignment with the malleolus and the first restraining bolt 118A could be off-center for selectively preventing pivoting. That is, the off-center restraining bolt(s) 118—regardless of specific configuration—could be manipulated by a prescriber or user to prevent pivoting of one or both of the lateral or medial upright extensions 20′ or 22′ with respect to the respective lateral or medial sidewalls 18 and 16 at a desired “restrict second direction pivoting” time, and could be manipulated to allow at least partial pivoting of one or both of the lateral or medial upright extensions 20′ or 22′ with respect to the respective lateral or medial sidewalls 18 and 16 at a desired “allow second direction pivoting” time. One of ordinary skill in the art will be able to provide one or more restraining bolts 118 having a desired type, location, and other physical configuration for a particular use environment of the external ankle brace 50′.

Regardless of how the restraining bolts 118 are configured and located on the various other components of the external ankle brace, it is contemplated that the restraining bolts 118 could be manipulated by any suitable party, at any desired time before, during, and/or after wear of the external ankle brace 50 and 50′, and for any desired reason. For example, the pivoting could be further restricted once an already tender ankle is further stressed, or the pivoting could be further permitted if a previously tender ankle responds well to light, restricted-pivoting duty. It is contemplated that one or more components of the external ankle brace 50 and 50′ could include a slot (e.g., a curved slot) within which a corresponding restraining bolt 118 can relatively travel or slide during use, in order to permit a limited amount of pivoting. It is also contemplated that one or more of the restraining bolt(s) could be a Chicago screw/bolt type, include a post and/or sleeve feature, or otherwise be configured to facilitate smooth rotation (and/or avoid wear) between two or more components of the external ankle brace 50 and 50′, whether or not they are permitted to selectively pivot relative to one another.

Via the aspects of the external ankle braces 50 and 50′ shown and described herein, a user can place the external ankle brace 50 and 50′ around an existing shoe (thus obviating the expense and inconvenience of special and/or mismatched shoes to accommodate an inside-the-shoe brace), tighten as desired, and accordingly selectively restrict movement of an ankle in a first direction and selectively permit movement of the ankle in a second direction (e.g., through selective use of the pivoting restriction schemes described above). The external ankle brace 50 also restricts rotation of the ankle (“windshield wiper” motion of the toes with a stationary heel, or vice versa). Accordingly, an ankle can receive a desired amount of support—capable of changing very quickly, even during a single wear session (e.g., an athletic or daily-activity event) via use of the restraining bolt(s) 118—and thus avoid initially or additionally injuring an ankle, foot, leg, or other portion of the wearer's body.

While aspects of this disclosure have been particularly shown and described with reference to the example aspects above, it will be understood by those of ordinary skill in the art that various additional aspects may be contemplated. For example, the specific methods described above for using the apparatus are merely illustrative; one of ordinary skill in the art could readily determine any number of tools, sequences of steps, or other means/options for placing the above-described apparatus, or components thereof, into positions substantively similar to those shown and described herein. In an effort to maintain clarity in the Figures, certain ones of duplicative components shown have not been specifically numbered, but one of ordinary skill in the art will realize, based upon the components that were numbered, the element numbers which should be associated with the unnumbered components; no differentiation between similar components is intended or implied solely by the presence or absence of an element number in the Figures. Any of the described structures and components could be integrally formed as a single unitary or monolithic piece or made up of separate sub-components, with either of these formations involving any suitable stock or bespoke components and/or any suitable material or combinations of materials. Padding or other cushioning material could be placed on any desired surface(s) of the components of the external ankle brace 50, 50′ to assist with force absorption, spacing, abrasion resistance, or for any other reason. A restraining bolt 118 could be selectively associated with only a chosen one of the lateral and medial upright extensions 20′ and 22′ to prevent with respect to the respective lateral and medial sidewall 18 and 16 at a desired “restrict second direction pivoting” time, with pivoting of the other of the lateral and medial upright extensions 20′ and 22′ being indirectly limited via the connection to the other of the lateral and medial upright extensions 20′ and 22′ through the upright connecting strap(s) 42. Any of the described structures and components could be disposable or reusable as desired for a particular use environment. Any component could be provided with a user-perceptible marking to indicate a material, configuration, at least one dimension, or the like pertaining to that component, the user-perceptible marking potentially aiding a user in selecting one component from an array of similar components for a particular use environment. A “predetermined” status may be determined at any time before the structures being manipulated actually reach that status, the “predetermination” being made as late as immediately before the structure achieves the predetermined status. The term “substantially” is used herein to indicate a quality that is largely, but not necessarily wholly, that which is specified—a “substantial” quality admits of the potential for some relatively minor inclusion of a non-quality item. Though certain components described herein are shown as having specific geometric shapes, all structures of this disclosure may have any suitable shapes, sizes, configurations, relative relationships, cross-sectional areas, or any other physical characteristics as desirable for a particular application. Any structures or features described with reference to one aspect or configuration could be provided, singly or in combination with other structures or features, to any other aspect or configuration, as it would be impractical to describe each of the aspects and configurations discussed herein as having all of the options discussed with respect to all of the other aspects and configurations. A device or method incorporating any of these features should be understood to fall under the scope of this disclosure as determined based upon the claims below and any equivalents thereof.

Embodiments include an external gauntlet ankle/foot orthosis which applies over the shoe providing ankle and foot stability prophylactically or following acute or chronic trauma. Embodiments relate to an orthotic that limits or prevents ankle inversion, eversion, dorsiflexion and plantar flexion and well as providing mid foot support.

Embodiments can minimize ankle inversion and eversion during physical activity and/or minimize ankle medial and later rotation during physical activity and/or minimize ankle plantar flexion and dorsiflexion during physical activity and/or provide stability to the mid foot in limiting pronation and supination of the foot.

Embodiments can include a foot/ankle orthotic that includes a lateral sidewall, a medial sidewall, a heel enclosed backing connecting the sidewalls, a lateral upright extension, a medial upright extension and a bottom strapping system connecting sidewalls. The lateral and medial upright extensions are attached to the sidewalls with an overlapping ankle joint off-set to accommodate for medial and lateral malleolus anatomical positioning. The lateral sidewall coincides with the outer or exterior portion of the foot/ankle and the medial sidewall coincides with the inner portion of the foot/ankle. The lateral upright extension coincides with the outer or exterior portion of the lower leg and the medial upright extension coincides with the inner portion of the lower leg. Lateral and medial extension walls are configured to rise above the ankle of the wearer of the orthotic by approximately 8-9 inches (from the bottom of the hinge to the top of the extension walls). When donned by the wearer, lateral and medial side walls also partially wrap over the top or dorsum of the foot leaving a gap of approximately 3 to 4 inches between the sidewalls. The width of the medial and lateral upright extensions is approximately 3-4 inches wide.

A feature of an embodiment is to have the securing mechanism include a hook and loop strap across the dorsal (top) of the foot. This Velcro securing strap is riveted to the in place on both the medial and later side walls. A D ring is utilized on the lateral fixation in which the Velcro strap can be fed through and secured back upon itself. The lateral and medial upright extensions are secured by two removable Velcro straps and D rings. Male component Velcro is adhesively attached to each upright and the female component Velcro strap can connect to the uprights are desired positions for appropriate fitting. As an option, the brace may also be applied with various types of athletic adhesive tape in conjunction with or instead of the Velcro strapping and D ring system.

Another feature of an embodiment is an overlapping ankle joint hinge to allow the ankle to move freely through plantar flexion and dorsiflexion. The overlapping ankle joint is located on the medial and lateral aspects of the gauntlet where the medial and lateral side bodies attach with the medial and lateral uprights respectively. The ankle joint hinge components are off set to produce a more anatomically correct gauntlet for a more fluid mobility.

Foam padding (approximately ¼ inch) is attached to the inside of both the medial and lateral uprights to provide additional comfort and protection for the wearer. The gauntlet is sized so that one size can fit multiple size shoes. A separate gauntlet is needed to accommodate both right and left ankles.

A sheet of vacuum formable thermoplastic large enough to cover the entire mold is cut and placed in an oven to be heated to a formable temperature. These are several types and thicknesses of plastic that may be used for this fabrication including orthotic grade polypropylene, polyethylene, and copolymer.

Other aspects, objects, and advantages can be obtained from a study of the drawings, the disclosure, and the appended claims.

We claim:

1. An external ankle brace for restricting movement of an ankle in at least one of a first direction or a rotation direction, and selectively permitting movement of the ankle in a second direction, wherein the external ankle brace is disposed on the exterior of an athletic shoe worn by a human at an end of a leg thereof, the shoe having a heel portion, a toe portion longitudinally spaced from the heel portion, a sole, and oppositely disposed sides, the external ankle brace comprising:
a lower leg portion that extends along a lower leg section of the human when the brace is worn by the human; and
a foot portion that interfaces with the shoe at locations where the foot of the human below a calf of the human on that leg is located, the foot portion including a rigid heel enclosure,
wherein the lower leg portion includes two uprights both selectively perpendicular to the rigid heel enclosure and pivotally attached to the foot portion,
wherein the foot portion includes a first subsection and a second subsection, the first subsection being configured to receive and at least partially encircle the heel portion of the athletic shoe and the human's foot, the second subsection having a medial sidewall and a lateral sidewall for collectively and concurrently at least partially encircling the sides of the athletic shoe concurrent with the first subsection connecting the medial and lateral sidewalls to collectively at least partially encircle the sides of the shoe and fully encircle the heel portion of the shoe, the lateral and medial sidewalls each extending from the first subsection toward a toe of a wearer's foot and each extending beyond a talus of the wearer's foot,
wherein the external ankle brace further comprises at least one lower fastening system comprising at least one lower connecting strap for connecting the lateral sidewall to the medial sidewall and extending underneath the sole of the shoe, and
wherein the external ankle brace further comprises at least one upper fastening system comprising an upper connecting strap for selectively connecting the lateral sidewall to the medial sidewall across the top of the shoe, wherein the lower leg portion is jointedly connected to the foot portion by respective ankle joints joining respective uprights to respective sidewalls.

2. The external ankle brace of claim 1, wherein the athletic shoe is a cleated shoe and the shoe is located in the external ankle brace with the upper fastening system and the lower fastening system extending over and below, respectively, the athletic shoe.

3. The external ankle brace of claim 2, wherein the human is wearing the athletic shoe.

4. The external ankle brace of claim 1, wherein there is no respective reinforcing strut in the respective uprights.

5. The external ankle brace of claim 1, wherein:
a subportion of the lower leg portion on the medial side of the brace extends downward over the foot portion in an overlapping manner so that a distance from a pivot axis of the respective ankle joint to an end of the subportion of the lower leg portion in the downward direction below a fastener that connects the upper strap to the foot section is about the same as a distance from the fastener to a front or rear end of the lower leg portion on the medial side in a horizontal direction.

6. The external ankle brace of claim 1, wherein:
the lower leg portion extends downward over the foot portion so as to have an overlapping subsection, a subsection of the foot portion that overlaps with the overlapping subsection is completely inboard of the overlapping subsection.

7. The ankle brace of claim 11, wherein:
the second subportion is a first sidewall portion and the third subportion is a second sidewall portion.

8. An external ankle brace for selectively restricting movement of an ankle in at least one of a first direction and a rotation direction, and selectively permitting movement of the ankle in a second direction, wherein the external ankle brace is disposed on the exterior of a shoe, the shoe having a heel portion, a toe portion longitudinally spaced from the heel portion, a sole, and oppositely disposed sides, the external ankle brace comprising:
a rigid heel enclosure having a rear portion and a forward portion, the rear portion configured to receive and at least partially encircle the heel portion of the shoe, the forward portion having a medial sidewall and a lateral sidewall for collectively and concurrently at least partially encircling the oppositely disposed sides of the shoe concurrent with the rear portion connecting the medial and lateral sidewalls, and for fully encircling the heel portion, of the shoe, the lateral and medial sidewalls each configured to extend from the rear portion toward a toe of a wearer's foot and to extend beyond a talus of the wearer's foot when the brace is worn over the shoe;
a lateral upright extension selectively perpendicular to the rigid heel enclosure and pivotally attached to the lateral sidewall, the lateral upright extension including a lateral reinforcing component;
a medial upright extension selectively perpendicular to the rigid heel enclosure and pivotally attached to the medial sidewall, the medial upright extension including a medial reinforcing component;
a lower fastening system comprising at least one lower connecting strap that is a synthetic strap for connecting the lateral sidewall to the medial sidewall and extending underneath the sole of the shoe; and
an upper fastening system comprising at least one upper connecting strap for selectively connecting the lateral sidewall to the medial sidewall across the top of the shoe.

9. The external ankle brace of claim 8, wherein:
the lateral upright extension is pivotally connected to the lateral sidewall by a restraining bolt, the lateral upright extension being able to pivot relative to the lateral sidewall; and
the lateral upright and the lateral sidewall is sized and dimensioned so that the pivot cannot be prevented upon the application of a plurality of secondary retraining bolts arrayed about a pivot axis established by the pivotally connection.

10. The external ankle brace of claim 8, wherein:
the lateral upright extension is pivotally connected to the lateral sidewall by a restraining bolt, the lateral upright extension being able to pivot relative to the lateral sidewall; and
the lateral upright and the lateral sidewall is sized and dimensioned so that the pivot is operable forward and a backward.

11. An external ankle brace, comprising:
a lower leg section configured to interface with a leg of a human at a lower leg thereof; and
a foot section configured to interface with a shoe worn on a foot of the human at locations where the foot of the human is located, wherein
the external ankle brace is configured to restrict movement of an ankle of the human in a first direction and permit movement of the ankle in a second direction, wherein said external ankle brace is configured to be disposed on the exterior of the shoe of the human, the shoe having a heel portion, a sole, an upper with oppositely disposed sides, and a toe section, the heel portion being at a rear of the shoe, and the toe section being at a front of the shoe,
the foot section is established by a portion that includes a first subportion, a second subportion and a third subportion, the first subportion of the foot section being configured to receive the rear of the shoe, the second subportion of the foot section extending away from the first subportion on a medial side of the foot when the brace is worn by the human, the third subportion of the foot section extending away from the first subportion on a lateral side of the foot when the brace is worn by the human,
the lower leg section includes a lateral upright extension that includes a portion that extends upward away from the foot section, the lateral upright extension being rotationally attached to the foot section,
the lower leg section includes a medial upright extension that includes a portion that extends upward away from the foot section, the medial upright extension being rotationally attached to the foot section, and
the external ankle brace further includes:
an upper fastening system comprising at least one synthetic connecting strap connecting said second subportion to said third subportion across the top of the shoe at a location that is in front of the lateral upright extension and medial upright extension;
a lower fastening system comprising at least one connecting strap attaching said second subportion to said third subportion underneath the sole of the shoe; and
a lower leg section fastening system comprising at least one connecting strap connecting said portion of said medial upright extension that extends upwardly away from the foot section with said portion of said lateral upright extension that extends upwardly away from the foot section.

12. The ankle brace of claim 11, wherein:
the at least one connecting strap of the lower fastening system includes only one connecting strap.

13. The ankle brace of claim 11, wherein:
the at least one connecting strap of the upper fastening system includes only one connecting strap.
